(19) United States
(12) Patent Application Publication
(10) Pub. No.: US 2005/0129751 A1
(43) Pub. Date: Jun. 16, 2005

(54) Drug delivery compositions and methods

(76) Inventors: Barry E. Rothenberg, Del Mar, CA (US); Fusun N. Zeytin, Del Mar, CA (US); Robert D. Fish, Tustin, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 10/738,604

(22) Filed: Dec. 16, 2003

(51) Int. Cl. A61K 39/00; A61K 9/127; A61K 9/22; A61K 9/16; A61K 9/50

(52) U.S. Cl. 424/450; 424/468; 424/184.1; 424/490

(57) ABSTRACT

Contemplated drug delivery systems allow controlled release of a drug in a manner that is independent of the physicochemical parameters of both the drug and its carrier. In one preferred aspect, the drug and the carrier are released from a second carrier that has a predefined release characteristics, which is predominantly determined by the physicochemical properties of the second carrier.

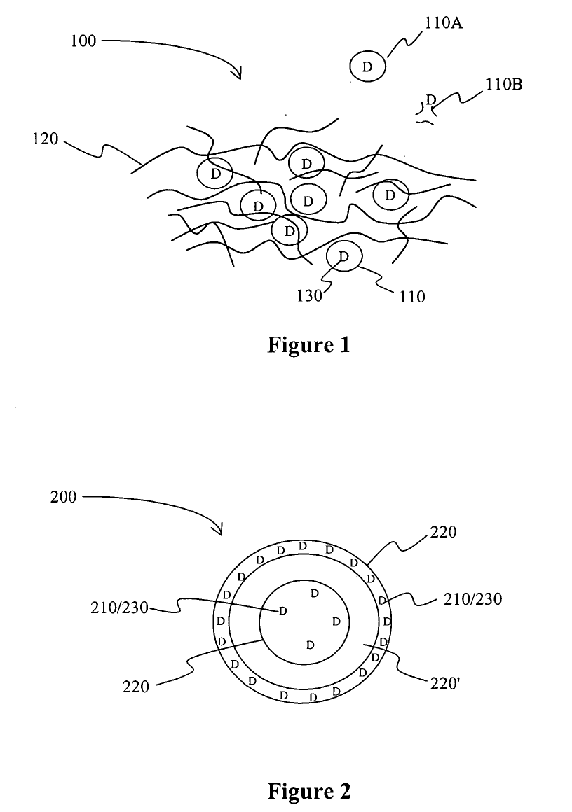

### FIELD OF THE INVENTION

Field of the invention is drug formulation, and especially as it relates to formulation of controlled drug delivery vehicles from a composite carrier.

### BACKGROUND OF THE INVENTION

Drug formulation for a specific drug is typically dependent on a combination of various parameters, including the physicochemical properties of a particular drug, route of administration of the drug, and the pharmacokinetic/pharmacodynamic properties of the drug. For example, where the drug is injected or orally administered in a liquid form, suitable formulations may be as simple as an aqueous solution of the drug. Similarly, where the drug is orally administered in a solid form, suitable formulations may be as simple as a powdered combination of the drug with a filler and an optional excipients.

However, where a drug is relatively unstable once the drug is administered, encapsulation with a solid or vesicular vehicle (e.g., liposome) is typically required to extend the serum half-life of the drug. Unfortunately, both the encapsulant and the drug will have distinct physicochemical characteristics, and the drug release kinetic/dynamic from the encapsulant is therefore in all but a few cases difficult to predict. Consequently, to achieve a specific drug release characteristic for a particular drug, considerable experimentation is often necessary to achieve a desired result. For example, where the encapsulant is a liposome, the range of liposome stability (and with this the dynamic of drug release) is in many formulations limited by the liposomal components and formulation method with the specific drug. On the other hand, where the encapsulant is a biodegradable or bioerodable biopolymer, stability can often be predetermined by the choice of polymer and method of fabrication. However, the process of encapsulation is typically not suitable for all drug molecules (e.g., heat-induced polymerization for peptide drugs, or chemically induced polymerization with chemically labile small molecule drugs).

Similarly, where it is preferred that the drug is continuously or discontinuously released from a carrier to achieve a particularly desirable schedule of administration, drug formulation for such purpose is frequently complicated by the physicochemical characteristics of the drug and/or the controlled release material. For example, where the drug carrier is an ion exchange resin, suitable drugs must be charged to interact with such resins. To overcome problems associated with charge, polarity, or other properties of a drug molecule, carriers with relatively weak carrier-drug interaction may be employed. For example, some biodegradable or bioerodable biopolymers may be used for controlled release. While such carriers typically provide a predictable drug release over a relatively long period of time, other problems arise. Among other things, and especially where the drug is a relatively large molecule (e.g., an antigen, enzyme, etc.) the drug may be partially exposed to the site of administration (typically blood/serum) and thus be partially degraded or immunogenic before the drug is completely released from the carrier.

Where modulated release (e.g., pulsatile release) is desired, difficulties are compounded as multiple layers have to be sequentially arranged in a micro-tablet or microcapsule. Similarly, multi-layered liposomes have not made a significant impact in drug formulation as various problems associated with manufacture of such liposomes often arise. Alternatively, as described in U.S. Pat. No. 6,214,377, two distinct carriers may be employed for a single drug, wherein the first carrier has a pulse-release characteristic and wherein the second carrier has a slow-release characteristic. While such approach at least partially simplifies pulsatile drug delivery, the same problems with drug-carrier interactions as described above remain.

Therefore, while there are numerous methods known in the art to formulate a drug into a controlled release formulation, various difficulties remain. Most significantly, drug formulation remains a predominantly empiric science due to drug- and carrier-specific physicochemical interactions. Thus, there is still a need to provide improved compositions and methods to formulate a drug in a controlled release formulation.

### SUMMARY OF THE INVENTION

The present invention is directed to compositions and methods of drug delivery system in which a secondary carrier with predefined release characteristics determines release of a drug that is coupled to a primary carrier in a manner that is substantially independent from the physicochemical characteristics of the drug.

Therefore, in one aspect of the inventive subject matter, a drug delivery system includes a first carrier having a first release rate relative to a drug, wherein the first carrier temporarily retains the drug. A second carrier having a second release rate relative to the drug, wherein the second carrier is admixed with and temporarily retains the first carrier while the first carrier retains the drug, wherein the second release rate is independent from the drug. Viewed from another perspective, a drug delivery system may comprise a drug that is temporarily coupled to a first carrier, and a second carrier in admixture with the first carrier, wherein the drug is released from the system while coupled to the first carrier.

In especially preferred drug delivery systems, the first carrier may be a liposome, a micelle, an ion-exchange resin, a biodegradable microcapsule, or a biodegradable dendrimer, and it is especially preferred that the first carrier encloses the drug to thereby temporarily retain the drug. Alternatively, the first carrier may also be covalently or ionically coupled to the drug. Preferred second carriers include biodegradable polymers, and/or biocompatible dissolving matrices, while particularly suitable drugs include various polypeptide drugs, small-molecule drugs, and/or antigens (for preventative and/or therapeutic vaccinations). The second release rate in especially contemplated delivery systems will typically be higher than the first release rate. Furthermore, first and second carriers may be arranged relative to each other (or relative to another material, e.g., biodegradable or bioerodable) in a manner such that delivery of the drug occurs discontinuously.

Consequently, a method of formulating a drug composition will include one step in which a drug is temporarily coupled to a first carrier. In another step, a second carrier is combined with the first carrier while the first carrier is coupled to the drug, wherein first and second carriers are selected such that release of the drug from the drug from the composition is determined by release of the first carrier from the second carrier. In further preferred methods, the step of temporarily coupling the drug to the first carrier comprises enclosing the first carrier into a liposome, a micelle, a biodegradable microcapsule or dendrimer, or a biocompatible dissolving matrix. Moreover, it is also contemplated that the step of temporarily coupling the drug to the first carrier comprises covalently or ionically coupling the drug to the first carrier.

In yet another aspect of the inventive subject matter, a method of determining proper dosage of a drug to a patient will include one step in which a drug delivery system is provided that comprises a first carrier having a first release rate relative to a tracer, wherein the first carrier temporarily retains the tracer, and a second carrier having a second release rate relative to the tracer, wherein the second carrier is admixed with and temporarily retains the first carrier while the first carrier retains the tracer, and wherein the second release rate is independent from the drug. In another step, the concentration of the tracer in the patient is determined, and in still another step, the concentration of the tracer is correlated with a release of the drug from the delivery system.

Various objects, features, aspects and advantages of the present invention will become more apparent from the following detailed description of preferred embodiments of the invention.

### DETAILED DESCRIPTION

The inventors generally contemplate that a drug delivery system includes a first carrier to which a drug is coupled (C1-D), and a second carrier (C2), wherein the C1-D and C2 are coupled or otherwise interconnected to each other such that release of the drug D (preferably in complex with C1) from C2 is determined by C1/C2 interaction, which is independent of physicochemical properties of drug D with its carrier C1. While not limiting to the inventive subject matter, it is typically preferred that D is released from C1 at a rate that is faster than a release rate of C1 from C2. Viewed from another perspective, the release from a drug into a system in most preferred aspects of the inventive subject matter is predominantly determined by interaction of C1 and C2, and substantially independent from interaction between D and C2. Particularly preferred carriers are sub-microscopic in size (i.e., less than 10 micron), and/or it is further especially preferred that the first carrier C1 (typically bound to the drug D) is molecularly interspersed with the second carrier C2.

The term “carrier” as used herein refers to a composition of matter that is intermixed with a drug and temporarily retains the drug, and further refers to one or more membranous vesicles (e.g., liposomes, transferosomes, etc.), but expressly excludes capsules or other materials that surround and enclose the drug, wherein the capsule and/or other material does not contain the drug.

As also used herein, the term “temporarily retains a/the drug” with respect to a carrier means that the carrier has an attractive or retaining physical (e.g., enclosing within a vesicular structure) and/or chemical interaction (e.g., via charge complementary interaction, hydrophilic or hydrophobic interaction, electrostatic interaction, etc.) with the drug such that the drug is coupled to or enclosed within the carrier for a predetermined period of time. Thus, a mixture of a drug with an inert substance that does not exhibit a physical and/or chemical interaction with the drug is not considered to temporarily retain the drug. Similarly, the term “temporarily retains a/the [first] carrier” with respect to a second carrier means that the second carrier has an attractive or retaining physical (e.g., enclosing within a vesicular structure) and/or chemical interaction (e.g., via charge complementary interaction, hydrophilic or hydrophobic interaction, electrostatic interaction, etc.) with the first carrier such that the first carrier is coupled to or enclosed within the second carrier for a predetermined period of time.

Contemplated First and Second Carriers

It is contemplated that all known pharmaceutically acceptable carriers are suitable for use in conjunction with the teachings presented herein, and it should be recognized that the first and second carriers may be chemically or structurally identical or distinct from each other. However, it is generally preferred that the first and second carriers are chemically and structurally distinct.

Therefore, in one aspect of the inventive subject matter, preferred first and/or second carriers include membranous vesicles that enclose the drug (which is typically dissolved in a solvent). Depending on the particular drug and the solvent used for the drug, suitable membranous vesicles may therefore be single- or multi-lamellar liposomes (which may or may not be inside-out-liposomes), nanosomes, or transferosomes. There are numerous liposome compositions and methods known in the art, and all of such compositions and methods are considered suitable for use herein. For example, contemplated compositions and methods are described in “Liposomes: A Practical Approach” by V. P. Torchilin et al.; Oxford University Press; 2nd edition (August 2003); (ISBN: 0199636540), or in “Liposomes: Rational Design” by Andrew S. Janoff; Marcel Dekker; (ISBN: 0824702255). Where appropriate, such liposomes may further include one or more selected ligands that may enhance the selectivity towards a specific target or set of targets. Exemplary protocols for preparation of such vesicles is described in U.S. Pat. No. 6,593,308 to Szoka J., or in the scientific literature (see e.g., G. Gregoriadis in “Use of Monoclonal Antibodies and Liposomes to Improve Drug Delivery. Present Status and Future Implications”, Drugs, 24, 261-266, (1982); or D. Papahadjopoulos, et al. in “Development of Liposomes as an Efficient Carrier System: New Methodology for Cell Targeting and Intracellular Delivery of Drugs and DNA”, Targeting of Drugs, (G. Gregoriadis, J. Senior and A. Trouet, eds), pp. 375-391. Plenum Press, New York, (1982); or E. Mayhew and D. Papahadjopoulos, “Therapeutic Applications of Liposomes”, Liposomes, (M. J. Ostro, ed.), pp. 289-341, Marcell Dekker, New York, (1983); or J. N. Weinstein and L. D. Leserman, “Liposomes as Drug Carriers in Cancer Chemotherapy”, Pharmacol. Ther., 24, 207-233, (1984)).

Alternatively, suitable first and/or second carriers also include materials that temporarily retain a drug via ionic interaction. There are numerous ion exchange resins for controlled drug release known in the art, and all of such resins are considered suitable for use herein. For example, where the drug is a cationic species, suitable ion exchange resins may include a sulfonic acid group (or modified sulfonic acid group) or a optionally modified carboxylic acid group on a physiologically acceptable scaffold. Similarly, where the drug is an anionic species, contemplated ion exchange resins may include amine-based groups (e.g., trimethylamine for strong anion exchange, or dimethylethanolamine for weak anion exchange). Suitable ion exchange resins for controlled drug release are described, for example, in “Evaluation of ion-exchange microspheres as carriers for the anticancer drug doxorubicin: in-vitro studies.” By Chen et al., in J Pharm Pharmacol. 1992 March;44(3):211-5; or in “Rate of release of organic carboxylic acids from ion-exchange resins” by Farag et al., in J Pharm Sci. 1988 October;77(10):872-5. It should be recognized that the drug may be coupled to the surface of the ion exchange resin or may be interspersed with the ion exchange resin (especially where the scaffold id biodegradable or bioerodable). Furthermore, it should be recognized that where the drug has a neutral charge, hydrophilic or hydrophobic interaction may be employed to temporarily retain the drug. Once more there are numerous hydrophilic and hydrophobic materials known in the art, and all of those are deemed appropriate in conjunction with the teachings presented herein.

In yet further contemplated aspects, a first and/or second carrier may also comprise a (nano)porous material that at least partially encloses the drug, and wherein the drug is eluted (i.e., via diffusion) from the nanoporous material over time. The inventors contemplate that such materials may be fabricated in numerous manners, including those known from the manufacture from molecular sieves. Alternatively, suitable (nano)porous materials may also be fabricated from a mixture of a removable (preferably polymeric) material and another (preferably polymeric) material. For example, removable materials include soluble (e.g., inorganic salts) or meltable materials (e.g., waxes, high melting point olefins, etc.), while the other materials is preferably a biodegradable or bioerodable polymer (see below). Further contemplated materials also include sol-gel materials (e.g., silicate based), or bio-organic polymeric materials (e.g., collagen or agarose matrix) that are well known in the art. Especially contemplated porous/matrix materials include those that form a gel in situ, and exemplary compositions and methods are described in U.S. Pat. No. 6,528,080. It should be recognized that the drug release rate in such materials can be controlled via pore size and frequency as well as via other chemical interactions with the material (e.g., ion exchange, or hydrophobic interaction properties).

Further especially suitable first and/or second carriers include biodegradable and/or bioerodable materials (and particularly polymers), and all of such known materials and mixtures comprising same are contemplated for use herein. For example, biodegradable materials include poly(lactides), poly(glycolides), collagens, poly(lactide-co-glycolides), poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polycyanoacrylates, poly(p-dioxanone), poly(alkylene oxalates), biodegradable polyurethanes, blends and copolymers thereof. Such materials may be configured as surface eroding structures, as a bulk structure, or as a structure that is formed in vivo. Exemplary materials and methods for preparation can be found in U.S. Pat. Nos. 4,938,763 and 5,324,519 to Dunn, U.S. Pat. Nos. 5,922,682 and 5,922,338 to Brich, or in U.S. Pat. No. 5,688,530 to Bodmer.

Alternatively, a drug may also be coupled to a macromolecular structure as a first carrier to form a covalent adduct, and it is generally contemplated that such adducts are in soluble form. For example, suitable macromolecular structures especially include hydrophilic polymers, and particularly polyethylene glycols, polyhydroxy acids, and oligopeptides. There are numerous manners of coupling polymers to a drug known in the art, and all of such manners are considered suitable for use herein. (see e.g., Kozlowski et al. in J. Control Release May 14, 2001;72(1-3):217-24). Where the drug is a nucleic acid (or a nucleic acid encoded by another nucleic acid), it is further preferred that the first carrier may also be a virus or virus particle. Moreover, the drug may also be formulated without a first carrier where the drug is in a microcrystalline form.

With respect to the relative amounts of first carrier (or C1-D) to the second carrier, it should be recognized that relative amounts of C1/C1-D to C2 will depend at least in part on desired amount of drug, but also on release kinetics of C1-D from C2, and C1 from D. For example, it is contemplated that the amount of C1-D is greater than the amount of C2 where relatively large quantities of drug are desired, and/or where release of D from C1-D is relatively slow. On the other hand, C1-D and C2 are approximately in the same proportions where release kinetics of C1-D from C2, and C1 from D are similar. In yet another example, the amount of C1-D is typically less than the amount of C2 where relatively small quantities of drug at low release rates (C1-D from D2) are desired. Therefore, preferred ratios of C1-D to C2 will be in the range of between about 1 to 100 and about 100 to 1.

It should further be recognized that the physical arrangement of C1-D and C2 relative to each other may vary, and will typically be depend on the particular chemical composition of C1 and/or C2. For example, where C1 is a liposome (enclosing the drug D), and where C2 is a biodegradable polymeric matrix, C2 will be in intimate admixture with C1-D. Similarly, where C1 is a biodegradable polymer (coupled to, or enclosing the drug D), and where C2 is a bioerodable polymeric matrix, C2 will be in intimate admixture with C1-D. On the other hand, more structures arrangements are also contemplated, and especially preferred structured arrangements include layered arrangements in which one C1-D/C2 admixture is enclosed or admixed with another C1-D/C2 admixture, wherein the amount of the drug D is different in one admixture as compared to the other admixture. Alternatively, or additionally, the relative quantities of C1-D to C2 may vary between the two admixtures. Such layered compositions are expected to exhibit complex drug release characteristics.

In further preferred aspects, it should be recognized that C1 may be selected such that the drug is released from C2 while the drug is coupled to C1 (i.e., as C1-D). Typically, such selection will predominantly be determined by the relative stability of C1-D and C2 in the environment of administration. For example, where the first carrier also acts as a drug targeting moiety (e.g., via liposome with a ligand on the surface), the liposome stability will be relatively high as compared to the stability of the second carrier. On the other hand, where the first carrier acts to protect the drug in the second carrier, the stability of the first carrier may lower than the stability of the second carrier.

Thus, and especially where the drug is released while bound to the first carrier, the release rate of the drug D will be substantially independent relative to the carrier C2. Consequently, once a second carrier with a desired release rate has been selected, any drug can be formulated with that second carrier in form of a C1-D complex, wherein the drug release is then independent from a potential interaction between the second carrier and the drug.

Contemplated Drugs

It should be appreciated that all known drugs are deemed suitable for use in conjunction with the teachings presented herein. Furthermore, it is contemplated that (where appropriate) two or more drugs may be formulated together to form contemplated controlled release formulations. In such formulations, it should be recognized that the first and second drugs may be coupled to the same first carrier or a different carrier. Suitable drugs may be characterized by their chemical structure or nature. Thus, in one aspect of the inventive subject matter, suitable drugs will include synthetic small-molecule drugs (i.e., with MW of less than 700), biological drugs (i.e., drugs isolated from a bacterium, yeast, cell, or organ, especially including recombinant polypeptides), and biosynthetic drugs (e.g., aptamers, antisense nucleic acid, siRNA, recombinant nucleic acid, nucleoside analogs, recombinant polypeptides, polypeptide drugs, antigens, etc).

Therefore, and viewed from another perspective, suitable drugs may also be characterized by their therapeutic activity, and particularly contemplated drugs include analgesics, anesthetics, anti-Parkinson's agents, anti-infectives, antibiotics, anticholinergics, anticoagulants, anticonvulsants, antidiabetic agents, antidyskinetics, antifibrotics, antifungal agents, antiglaucoma agents, anti-inflammatory agents, antineoplastics, antiosteoporotics, antipyretics, antiseptics/disinfectants, antithrombotics, bone resorption inhibitors, calcium regulators, cardioprotective agents, cardiovascular agents, central nervous system stimulants or depressants, cholinesterase inhibitors, contraceptives, dopamine receptor agonists, erectile dysfunction agents, fertility agents, gastrointestinal agents, gout agents, hormones, hypnotics, immunomodulators, immunosuppressives, keratolytics, motion sickness agents, muscle relaxants, obesity agents, ophthalmic agents, parasympatholytics, parasympathomimetics, prostaglandins, psychotherapeutic agents, respiratory agents, sclerosing agents, sedatives, skin and mucous membrane agents, smoking cessation agents, sympatholytics, synthetic antibacterial agents, ultraviolet screening agents, urinary tract agents, vaginal agents, vasodilators, and all reasonable combinations thereof

Thus, exemplary anti-bacterial compounds include 4-sulfanilamidosalicylic acid, acediasulfone, amfenac, amoxicillin, ampicillin, apalcillin, apicycline, aspoxicillin, aztreonam, bambermycin(s), biapenem, carbenicillin, carumonam, cefadroxil, cefamandole, cefatrizine, cefbuperazone, cefclidin, cefdinir, cefditoren, cefepime, cefetamet, cefixime, cefinenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, ciprofloxacin, clinafloxacin, cyclacillin, enoxacin, epicillin, flomoxef, grepafloxacin, hetacillin, imipenem, lomefloxacin, lymecycline, meropenem, moxalactam, mupirocin, nadifloxacin, norfloxacin, panipenem, pazufloxacin, penicillin N, pipemidic acid, quinacillin, ritipenem, salazosulfadimidine, sparfloxacin, succisulfone, sulfachrysoidine, sulfaloxic acid, teicoplanin, temafloxacin, temocillin, ticarcillin, tigemonam, tosufloxacin, trovafloxacin, vancomycin, etc. A drug of particular interest is an anti-Mycoplasma drug for treating tuberculosis, because the long course of treatment and generally poor patient compliance.

Exemplary anti-fungal compounds include amphotericin B, azaserine, candicidin(s), lucensomycin, natamycin, nystatin, etc. Exemplary anti-neoplastic compounds include 6-diazo-5-oxo-L-norleucine, azaserine, carzinophillin A, denopterin, edatrexate, eflomithine, melphalan, methotrexate, mycophenolic acid, podophyllinic acid 2-ethylhydrazide, pteropterin, streptonigrin, Tomudex.RTM. (N-((5-(((1,4-Dihydro-2-methyl-4-oxo-6-quinazolinyl)methyl)methylamino)-2-thienyl)carbonyl)-L-glutamic acid), ubenimex, etc. Exemplary anti-thrombotic compounds include argatroban, iloprost, lamifiban, taprostene, tirofiban, etc. Exemplary immunosuppressive compounds include bucillamine, mycophenolic acid, procodazole, romurtide, ubenimex, etc.

Exemplary NSAID compounds suitable for use herein include 3-amino-4-hydroxybutyric acid, aceclofenac, alminoprofen, bromfenac, bumadizon, carprofen, diclofenac, diflunisal, enfenamic acid, etodolac, fendosal, flufenamic acid, gentisic acid, meclofenamic acid, mefenamic acid, mesalamine, niflumic acid, olsalazine oxaceprol, S-adenosylmethionine, salicylic acid, salsalate, sulfasalazine, tolfenamic acid, etc.

Contemplated Uses and Methods

Contemplated drug delivery systems may be employed in numerous manners, and the particular composition of the delivery system will at least in part determine the particular use. For example, where the first and second carriers are in a liquid form, or where the first and second carriers are relatively small particles (e.g., less than 50 micron), contemplated may be injected. On the other hand, it is also contemplated that the delivery systems according to the inventive subject matter may also be administered in oral dosage forms. In yet further preferred aspects, contemplated delivery systems may also be implanted. Thus, contemplated delivery systems may be useful in various stages of drug discovery, development, or actual formulation of a drug.

In one particularly preferred aspect, the inventors contemplate that the delivery system according to the inventive subject matter may be employed to establish a proper drug dosing for a particular drug to a patient. For example, it is well known in the art that the pharnacokinetic and/or pharmacodynamic of a drug varies significantly from patient to patient. Therefore, a delivery system comprising a tracer may be administered first to a patient in which the concentration and/or distribution of the tracer is followed over time. Among numerous alternatives, the tracer may be a metabolically inactive compound that is chemically at least somewhat related to the drug (e.g., an L-nucleoside, where the drug is a D-nucleoside analog), wherein the metabolically inactive compound is coupled to the first carrier. The release kinetic and/or dynamic of the tracer may then be determined by following the amounts of the tracer in the patient (e.g., in serum, urine, etc.). Based on such determination, a drug may then be formulated into contemplated drug delivery systems to achieve a predetermined, patient-specific drug release.

Alternatively, suitable tracers may include otherwise detectable molecules which may or may not chemically resemble the drug that is to be administered. For example, contemplated tracers include fluorescent compounds (e.g., fluorescein), small molecules for which standard assay procedures are readily available (e.g., biotin, or biotinylated drugs), and so forth. In yet further contemplated aspects, the tracer may be a modified form of the drug that is coadministered with the drug (e.g., isotopically labeled drug, or biotinylated drug).

For example, a desired release rate for a drug may be achieved by using contemplated drug delivery system and a calibration curve in which the release rate of a particular drug from the delivery system in plotted as a function of the release rate of the tracer from the delivery system. Thus, contemplated delivery systems may be employed to customize drug delivery to a patient according to the patient's individual metabolic and/or pharmacokinetic/dynamic characteristics. Therefore, particularly preferred methods of determining a proper dosage of a drug to a patient will include a step in which a drug delivery system comprises a first carrier having a first release rate relative to a tracer, wherein the first carrier temporarily retains the tracer, and a second carrier having a second release rate relative to the tracer, wherein the second carrier is admixed with and temporarily retains the first carrier while the first carrier retains the tracer, and wherein the second release rate is independent from the drug. In a further step, the concentration of the tracer is determined in the patient, and the concentration of the tracer is correlated with a release of the drug from the delivery system.

Consequently, a method of formulating a drug composition may include one step in which a drug is temporarily coupled to a first carrier. In another step, a second carrier is combined with the first carrier while the first carrier is coupled to the drug, wherein first and second carriers are selected such that release of the drug from the drug from the composition is determined by release of the first carrier from the second carrier. As described above, it is especially preferred that the step of temporarily coupling the drug to the first carrier includes a step of enclosing the first carrier into a liposome, a micelle, a biodegradable microcapsule or admixture, a biodegradable dendrimer, or a biocompatible dissolving matrix. Alternatively, or additionally, the drug may also be covalently coupled to the carrier.

In such methods, the second carrier is preferably a biodegradable polymer, or a biocompatible dissolving matrix, while the drug may be a polypeptide drug, a small-molecule drug, or an antigen, and the release of the drug from the drug from the drug composition is slower than a release of the drug from the first carrier once the first carrier is released from the second carrier.

Therefore, contemplated drug delivery systems will preferably include a first carrier having a first release rate relative to a drug, wherein the first carrier temporarily retains the drug, and a second carrier having a second release rate relative to the drug, wherein the second carrier is admixed with and temporarily retains the first carrier while the first carrier retains the drug, and wherein the second release rate is independent from the drug. Typically (but not necessarily), the first carrier will enclose the drug to thereby temporarily retain the drug, wherein the second release rate is higher than the first release rate. Therefore, particularly suitable first carriers will include liposomes, a micelle, an ion-exchange resin, a biodegradable microcapsule, and/or a biodegradable dendrimer, and preferred second carriers will include a biodegradable polymer, or a biocompatible dissolving matrix.

Further preferred delivery systems will also include those in which the first carrier and the second carrier are arranged relative to each other to effect discontinuous drug delivery. For example, a layered arrangement may be employed to achieve pulsed administration of the drug or administration with increasing and/or decreasing drug release.

In still further preferred configurations and methods, a drug delivery system includes a drug that is temporarily coupled to a first carrier (e.g., liposome, micelle, ion-exchange resin, biodegradable microcapsule, or biodegradable dendrimer), and further includes a second carrier (e.g., biodegradable polymer, or biocompatible dissolving matrix) in admixture with the first carrier, wherein the drug is released from the system while at least some of the drug is coupled to the first carrier. Preferred drugs include various polypeptide drug (e.g., various interferons and fragments thereof), a small-molecule drug, or an antigen (e.g., viral epitopes).

All patents, published patent applications, and printed publications noted in the present application are specifically incorporated by reference herein.

Thus, specific embodiments and applications of drug delivery compositions and methods have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A drug delivery system, comprising:
a first carrier having a first release rate relative to a drug, wherein the first carrier temporarily retains the drug;
a second carrier having a second release rate relative to the drug, wherein the second carrier is admixed with and temporarily retains the first carrier while the first carrier retains the drug; and
wherein the second release rate is independent from the drug.

2. The drug delivery system of claim 1 wherein the first carrier encloses the drug to thereby temporarily retain the drug.

3. The drug delivery system of claim 1 wherein the second release rate is higher than the first release rate.

4. The drug delivery system of claim 1 wherein the first and second carriers are in an injectable preparation.

5. The drug delivery system of claim 1 wherein the first carrier is a liposome, a micelle, an ion-exchange resin, a biodegradable microcapsule, or a biodegradable dendrimer.

6. The drug delivery system of claim 1 wherein the second carrier is a biodegradable polymer, or a biocompatible dissolving matrix.

7. The drug delivery system of claim 1 wherein the drug is a polypeptide drug, a small-molecule drug, or an antigen.

8. The drug delivery system of claim 1 wherein the first carrier and the second carrier are arranged relative to each other such that delivery of the drug occurs discontinuously.

9. A drug delivery system comprising a drug temporarily coupled to a first carrier, and a second carrier in admixture with the first carrier, wherein the drug is released from the system while coupled to the first carrier.

10. The drug delivery system of claim 9 wherein the first carrier is a liposome, a micelle, an ion-exchange resin, a biodegradable microcapsule, or a biodegradable dendrimer.

11. The drug delivery system of claim 9 wherein the first carrier is covalently coupled to the drug.

12. The drug delivery system of claim 9 wherein the first carrier is in admixture with the second carrier.

13. The drug delivery system of claim 9 wherein the second carrier is a biodegradable polymer, or a biocompatible dissolving matrix.

14. The drug delivery system of claim 9 wherein the drug is a polypeptide drug, a small-molecule drug, or an antigen.

15. The drug delivery system of claim 9 wherein the first carrier and the second carrier are arranged relative to each other such that delivery of the drug occurs discontinuously

16. A method of formulating a drug composition, comprising:
temporarily coupling a drug to a first carrier;
combining a second carrier with the first carrier while the first carrier is coupled to the drug; and
wherein first and second carriers are selected such that release of the drug from the drug from the composition is determined by release of the first carrier from the second carrier.

17. The method of claim 16 wherein the step of temporarily coupling the drug to the first carrier comprises enclosing the first carrier into a liposome, a micelle, a biodegradable microcapsule, a biodegradable dendrimer, or a biocompatible dissolving matrix.

18. The method of claim 16 wherein the step of temporarily coupling the drug to the first carrier comprises covalently coupling the drug to the first carrier.

19. The method of claim 16 wherein the second carrier is a biodegradable polymer, or a biocompatible dissolving matrix.

20. The method of claim 16 wherein the drug is a polypeptide drug, a small-molecule drug, or an antigen.

21. The method of claim 16 wherein the release of the drug from the drug from the drug composition is slower than a release of the drug from the first carrier once the first carrier is released from the second carrier.

22. The method of claim 16 wherein the drug comprises an agent for treating Mycoplasma infection.

23. A method of determining proper dosage of a drug to a patient comprising:
providing a drug delivery system comprising a first carrier having a first release rate relative to a tracer, wherein the first carrier temporarily retains the tracer, and a second carrier having a second release rate relative to the tracer, wherein the second carrier is admixed with and temporarily retains the first carrier while the first carrier retains the tracer, and wherein the second release rate is independent from the drug;
determining a concentration of the tracer in the patient; and
correlating the concentration of the tracer with a release of the drug from the delivery system.

24. The method of claim 23 wherein the second release rate is higher than the first release rate.

25. The method of claim 23 wherein the first carrier is a liposome, a micelle, an ion-exchange resin, a biodegradable microcapsule, or a biodegradable dendrimer, and wherein the second carrier is a biodegradable polymer, or a biocompatible dissolving matrix.

26. The method of claim 23 wherein the drug is a polypeptide drug, a small-molecule drug, or an antigen.
